(19) United States
(12) Patent Application Publication
(10) Pub. No.: US 2022/0238181 A1
(43) Pub. Date: Jul. 28, 2022

(54) CRISPR GUIDE SELECTION

(71) Applicant: Recursion Pharmaceuticals, Inc., Salt Lake City, UT (US)

(72) Inventors: James JENSEN, Centerville, UT (US); Timothy DAHLEM, Salt Lake City, UT (US); Sarah HUGO, Farmington, UT (US); Jacob COOPER, Sandy, UT (US); Spencer SCHREIER, Cottonwood Heights, UT (US); Ian QUIGLEY, Salt Lake City, UT (US); Imran HAQUE, Salt Lake City, UT (US); Nathan LAZAR, Salt Lake City, UT (US); Alison GARDNER, Farmington, UT (US); Ben BANOWSKY, Salt Lake City, UT (US); August ALLEN, Salt Lake City, UT (US)

(73) Assignee: Recursion Pharmaceuticals, Inc., Salt Lake City, UT (US)

(21) Appl. No.: 17/585,660

(22) Filed: Jan. 27, 2022

Related U.S. Application Data
(60) Provisional application No. 63/142,342, filed on Jan. 27, 2021.

(51) Int. Cl. G16B 20/20; C12N 9/22; C12N 15/10; C12N 15/113; G16B 30/10

(52) U.S. Cl. CPC G16B 20/20; C12N 9/22; C12N 15/102; C12N 2320/11; G16B 30/10; C12N 2310/20; C12N 15/113

(57) ABSTRACT

A system for selecting CRISPR guides for knocking out one or more target genes in a target cell from a multiplicity of candidate guides comprises a memory and a processor. The processor determines whether the candidate guide meets a plurality of thresholds. The thresholds are associated with: a transcript support level; targeting a consensus sequence of a target gene; which exon of the target gene is targeted; targeting of a primary transcript, targeting of a common isoform; a precomputed prediction of editing outcomes; mapping to an expressed sequence; fraction of gene expression attributable to targeted transcripts; a common SNP overlap threshold; which exon of the target gene is targeted; overlap of a selected guide; predicted frameshift percentage; maximum and minimum GC content; off target score; where a coding sequence is targeted. In response to meeting the thresholds, the processor selects the candidate guide as a selected CRISPR guide.

### CROSS-REFERENCE TO RELATED APPLICATIONS

This application claims priority to and benefit of the provisional patent application, Ser. No. 63/142,342, Attorney Docket Number R2020.0009.US1, entitled “CRISPR GUIDE SELECTION,” with filing date Jan. 27, 2021 assigned to the assignee of the present application, which is herein incorporated by reference in its entirety.

### BACKGROUND

Clustered Regularly Interspaced Short Palindromic Repeats (CRISPR) technology has emerged as a preferred method for genetic screening. Guide sequences, also referred to as “CRISPR-Cas guide sequences,” or “CRISPR guide,” or simply as “guides,” by those of ordinary skill in the art are typically used to assist in discovery of valid gene knockout-dependent phenotypes. A CRISPR system using S. pyogenes Cas 9 requires a guide sequence matching a 20-base-pair segment of a gene followed by a NGG protospacer adjacent motif (PAM) sequence. A given gene may contain hundreds or thousands of such sequences that potentially could be targeted. Some of these sequences may be much more effective that other in several aspects: how frequently they induce frameshift mutations, ho completely those mutations abrogate the gene's function, and to what extent they cause off-target effects by binding or cutting elsewhere in the genome or by disrupting regulator elements that control expression of other genes.

### SUMMARY

In certain respects, the technology described herein employs transcript annotations, including but not limited to Ensembl transcription annotation to identify and rank coding sequences. In certain respects, the technology described herein employs a knockout model that is trained on data obtained from guide sequences, genes, and cell types. Features relate to sequence identity, composition, and position within a gene's coding sequence, overlap with epigenetic features, regulatory elements, and common polymorphisms, expression of the gene and of the transcripts targeted by the guide, and scores from tools that predict on-target efficacy, including but not limited to public tools such as FORECasT, Azimuth, and DeepCRISPR. The guide selection methods of the technology described herein correlate well with observed knockouts.

In certain embodiments, the (candidate guide identification) process comprises identifying target coding sequences and evaluating the biological relevance of the target coding sequences. In certain embodiments, the coding sequences are ranked according to one more characteristics that are likely bestow biological relevance. In certain embodiments, one or more of the coding sequence characteristics is predicted, for example by comparison to a database of coding sequences. In certain embodiments, one or more of the coding sequence characteristics is measured, for example by comparison to a set of coding sequences expressed by a tissue or cell type of interest, or determined to be critical to a phenotype of a tissue or cell type.

In certain embodiments, the ranking process comprises passing target coding sequences through one or more filters, each filter designed to distinguish coding sequences that satisfy selection criteria and reject coding sequences that do not. In certain embodiments, the filters are initially set to stringent thresholds, then progressively relaxed until a desired number of guides has been selected from a set of candidate guides. In certain embodiments, the filters are relaxed in a particular order and/or in particular increments. In certain embodiments, the ranking process comprises “nested” filters. That is, filters are applied in order and more deeply nested filters relaxed earlier in the selection procedure that less deeply nested filters. In certain embodiments, the filters can be performed in any order. In certain embodiments, the filters are performed in the order as set forth herein.

The technology described herein provides a method of selecting one or more CRISPR-Cas system guide sequences for generating loss-of-function mutations in coding sequences of target genes in a cell, which comprises one or more steps to identify and/or select guides from guide candidates that are biologically functional to generate one or more knock out mutations in one or more genes or coding sequences in a cell.

In certain embodiments, the guide selection process employs a frameshift prediction model to identify knockout targets for a particular cell type, tissue type, or phenotype of interest. In certain embodiments, the guide selection process employs selection criteria to identify and optionally rank gene targets according to gene expression characteristics. In certain embodiments, the guide selection process employs guide selection criteria to identify and optionally rank target sequences for frameshift efficiency. In certain embodiments, the guide selection process employs selection criteria to identify and optionally rank targets sequences according to uniqueness and/or dissimilarity to non-target sequences in the genome.

Criteria for guide selection include, without limitation, transcript support level; targeting of a consensus coding sequence; targeting a coding sequence not within the first coding exon, targeting a MANE transcript; targeting a principal transcript (e.g., a transcript with a low APPRIS score), whether there is a precomputed prediction of editing outcomes (e.g., FORECasT), whether the coding sequence is observed to be expressed, the fraction of gene expression attributable to transcripts comprising the targeted coding sequence, whether there is overlap with a common sequence polymorphism (e.g., a SNP), limiting the number of guides selected for an exon, minimizing overlap with other guides that target an exon, the predicted or measured rate at which a guide induces a frameshift mutation, a GC fraction greater than a selected threshold, a GC fraction less than a selected threshold, low off-target activity, and position along a coding sequence.

In certain embodiments, there is a hierarchy of guide selection criteria. The hierarchy provides for increased weight or stringency to be applied for selection criteria which have greater impact on guide success. The hierarchy may be user specified and/or determined experimentally. In certain embodiments, there is a hierarchy of two or more guide selection criteria, i.e., criteria are ranked by significance and when selection criteria are relaxed, less significant criteria are relaxed before more significant criteria.

In certain embodiments, there is an equivalence of guide selection criteria. Such equivalence provides for similar or equal weight to be applied for selection criteria. The equivalence may be user specified and/or determined experimentally. In certain embodiments, there is an equivalence of two or more guide selection criteria, i.e., certain criteria are ranked the same or similarly by significance and when the selection criteria are relaxed, the criteria that are ranked the same or similarly are relaxed together.

The technology described herein further comprehends a computer system for identifying one or more unique target sequences, e.g., in a genome, such as a genome of a eukaryotic organism, the system comprising: a.) a memory unit configured to receive and/or store sequence information of the genome; and b.) one or more processors alone or in combination programmed to perform a herein method of identifying one or more unique target sequences (e.g., locate a CRISPR motif, analyze a sequence upstream of the CRISPR motif to determine if the sequence occurs elsewhere in the genome, analyze a sequence upstream of the CRISPR motif to determine whether it meets selection criteria set forth herein, and select the sequence.

In another aspect, the technology described herein provides a guide library made using the methods as described herein. In a further aspect, the technology described herein provides a guide library comprising guide sequences to one or more target regions in one or more exons of one or more target genes, wherein individual guide sequences in the library are included based on optimization of an off-target avoidance score and an on-target efficiency score, and optionally, by the presence of a protein domain in the target region. In one embodiment, the exons are selected based on tissue-specific expression data to select exons with higher expression. In another embodiment, the off-target avoidance score is determined by taking the sum of a cutting frequency determination score for each off-target side identified in an exome of the one or more target genes. In another embodiment, the on-target efficiency is determined by use of a classifier applied to local sequence preferences learned from saturation mutagenesis studies. In another embodiment, the classifier is a boosted regression tree classifier. Other embodiments provide guide sequences that exclude guide sequences targeting homopolymer regions, targeting the last exon in a coding region, include target regions with transcriptional terminators, or a combination thereof. In still further embodiments, the guide sequences are full length guide sequences, truncated guide sequences, full length sgRNA sequences, truncated sgRNA sequence, or E+F sgRNA sequences, or the guide sequences are RNA, DNA, DNA-RNA hybrid, chemically modified, or a combination thereof.

In another aspect, the technology described herein provides a composition comprising a population of cells and a guide sequence library as described herein, where each of the cells contains one or more of the guide sequences and thus the guides sequences of the library are integrated into the population of cells. In one embodiment, the population of cells is a eukaryotic population of cells.

In another aspect, the technology described herein provides a kit comprising a guide sequence library as described herein, and/or a composition as described herein.

### BRIEF DESCRIPTION OF DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like items are labeled with like item numbers.

FIGS. 1A-1C depict head-to-head guide design comparisons of a plurality of guides selected according to embodiments described herein.

FIGS. 2A-2E depict some example aspects of a recursive procedure for CRISPR guide selection, according to various embodiments.

FIG. 3 illustrates components of an example computer system, with which or upon which, various embodiments may be implemented.

FIGS. 4A-4B illustrate a flow diagram of an example method of CRISPR guide selection, in accordance with various embodiments.

### DESCRIPTION OF EMBODIMENTS

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Overview of Discussion

With CRISPR technology, guides that efficiently cause full functional knockout with low off-target effect offer the best chance for discovery of valid gene knockout-dependent phenotypes. Thus, guide selection can have a large impact on results achieved. The technology described herein provides a guide selection method involving candidate identification by one or more criteria, knockout (frameshift) prediction, and iterative guide selection algorithms.

Description will begin with a discussion of notation and nomenclature, followed by a description of an analysis of coding sequences for guide selection using Ensembl, a genome browser for vertebrate genomes that supports research in comparative genomics, evolution, sequence variation and transcriptional regulation. Description proceeds with discussion of several figures which provide head-to-head guide design comparisons of a plurality of guides selected according to embodiments described herein. A recursive procedure for CRISPR guide selection, according to various embodiments. An example computer system is then described, with which or upon which, various embodiments may be implemented. Finally, a flow diagram of an example method of CRISPR guide selection is described. The method of the flow diagram may be implemented with a computer system such as the described computer system.

### Notation and Nomenclature

Unless defined otherwise, technical and scientific terms used herein have the same meaning as commonly understood by one of ordinary skill in the art to which this disclosure pertains.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed technology. It is to be understood that the value to which the modifier “about” or “approximately” refers is itself also specifically, and preferably, disclosed.

Some portions of the detailed descriptions which follow are presented in terms of procedures, logic blocks, processes, modules and other symbolic representations of operations on data bits within a computer memory. These descriptions and representations are the means used by those skilled in the data processing arts to most effectively convey the substance of their work to others skilled in the art. In the present application, a procedure, logic block, process, module, or the like, is conceived to be one or more self-consistent procedures or instructions leading to a desired result. The procedures are those requiring physical manipulations of physical quantities. Usually, although not necessarily, these quantities take the form of electrical or magnetic signals capable of being stored, transferred, combined, compared, and otherwise manipulated in an electronic device/component.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “determining,” “selecting,” “identifying,” “sequencing,” “synthesizing,” “storing,” “discarding,” “keeping,” “rejecting,” “adjusting,” or the like, refer to the actions and processes of an electronic device or component such as: a processor, a controller, a memory, a computer system or component(s) thereof, or the like, or a combination thereof. The electronic device/component manipulates and transforms data represented as physical (electronic and/or magnetic) quantities within the registers and memories into other data similarly represented as physical quantities within memories or registers or other such information storage, transmission, processing, or display components.

Embodiments described herein may be discussed in the general context of computer/processor executable instructions residing on some form of non-transitory computer/processor readable storage medium, such as program modules or logic, executed by one or more computers, processors, or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example hardware described herein may include components other than those shown, including well-known components.

The techniques described herein may be implemented in hardware, or a combination of hardware with firmware and/or software, unless specifically described as being implemented in a specific manner. Any features described as modules or components may also be implemented together in an integrated logic device or separately as discrete but interoperable logic devices. If implemented in software, the techniques may be realized at least in part by a non-transitory computer/processor-readable storage medium comprising computer/processor-readable instructions that, when executed, cause a processor and/or other components of a computer or electronic device to perform one or more of the methods described herein. The non-transitory computer/processor-readable data storage medium may form part of a computer program product, which may include packaging materials.

The non-transitory processor readable storage medium (also referred to as a non-transitory computer readable storage medium) may comprise random access memory (RAM) such as synchronous dynamic random access memory (SDRAM), read only memory (ROM), non-volatile random access memory (NVRAM), electrically erasable programmable read-only memory (EEPROM), Flash memory, compact discs, digital versatile discs, optical storage media, magnetic storage media, hard disk drives, other known storage media, and the like. The techniques additionally, or alternatively, may be realized at least in part by a processor-readable communication medium that carries or communicates code in the form of instructions or data structures and that can be accessed, read, and/or executed by a computer or other processor.

The various illustrative logical blocks, modules, circuits and instructions described in connection with the embodiments disclosed herein may be executed by one or more processors, such as host processor(s) or core(s) thereof, digital signal processors (DSPs), general purpose microprocessors, application specific integrated circuits (ASICs), application specific instruction set processors (ASIPs), field programmable gate arrays (FPGAs), graphics processing unit (GPU), microcontrollers, or other equivalent integrated or discrete logic circuitry. The term “processor” or the term “controller” as used herein may refer to any of the foregoing structures or any other structure suitable for implementation of the techniques described herein. In addition, in some aspects, the functionality described herein may be provided within dedicated software modules or hardware modules configured as described herein. Also, the techniques, or aspects thereof, may be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a plurality of microprocessors, one or more microprocessors in conjunction with an ASIC or DSP, or any other such configuration or suitable combination of processors.

### Analysis of Coding Sequences for Guide Selection

Ensembl is a genome browser for vertebrate genomes that supports research in comparative genomics, evolution, sequence variation and transcriptional regulation. Ensembl annotates genes, computes multiple alignments, predicts regulatory function and includes disease data. Ensembl tools include BLAST, BLAT, BioMart and the Variant Effect Predictor (VEP) for all supported species.

Ensembl annotation of a genome starts from targeted species-specific alignment of proteins to the genome and prediction of transcript structure for the protein on the genome. If a targeted structure is absent from the available sequence information, proteins from closely related species are used to build a transcript structure. The Ensembl annotation process includes alignment of species-specific cDNA and EST sequences to the genome. Where cDNA alignments overlap predicted transcripts, any non-translated region from the cDNA is spliced onto the transcript prediction as UTR. A maximum number of guides per exon for each candidate guide can be initialized at 201-1, and a maximum overlap can be initialized at 201-2.

Ensembl annotation includes automated procedures for non-coding RNAs (ncRNAs), including transfer RNA (tRNA), transfer RNA located in the mitochondrial genome (Mt-tRNA), ribosomal RNA (rRNA), small cytoplasmic RNA (scRNA), small nuclear RNA (snRNA), small nucleolar RNA (snoRNA), microRNA precursors (miRNA), miscellaneous other RNA (misc_RNA), and long intergenic non-coding RNAs (lincRNA). lincRNA annotation is specialized. Regions of chromatin methylation (H3K4me3 and H3K36me3) outside known protein-coding loci are identified, then cDNAs which overlap with H3K4me3 or H3K36me3 features are identified as candidate lincRNAs. Protein encoding potential is evaluated and any candidate lincRNA containing a substantial open reading frame (ORF) covering 35% or more of its length and containing PFAM/tigrfam protein domains is rejected.

A conventional standard reference human assembly sequence is the Genome Reference Consortium Human genome build 38 (GRCh38). GRCh38/hg38 is the assembly of the human genome released December of 2013, that uses alternate or ALT contigs to represent common complex variation, including HLA loci. GRCh38 is not from one individual's genome sequence but is built from reference sequences of different individuals. GRCh38 includes significant improvements in the representation of alternate haplotypes, i.e., regions that are sometimes dramatically different in different populations. Representation of these alternate haplotypes has a significant impact on the ability to detect and analyze genomic variation that is specific to populations that carry alternate haplotypes. GRCh38 advantageously allows accounting for regions of genomic variation, including to select or to avoid regions of variation. For example, in selecting coding sequences as generally useful knockout targets, it can be advantageous to avoid regions of variability. In selecting coding sequences for a particular subject population, it may be advantageous to select knockout targets specific to that subject population.

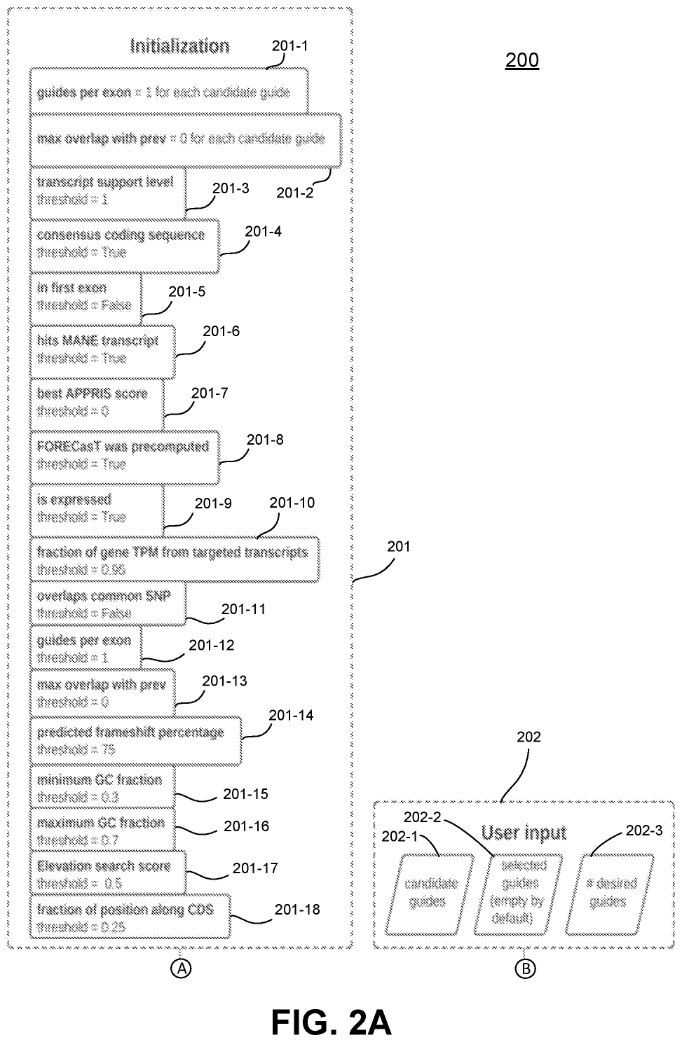

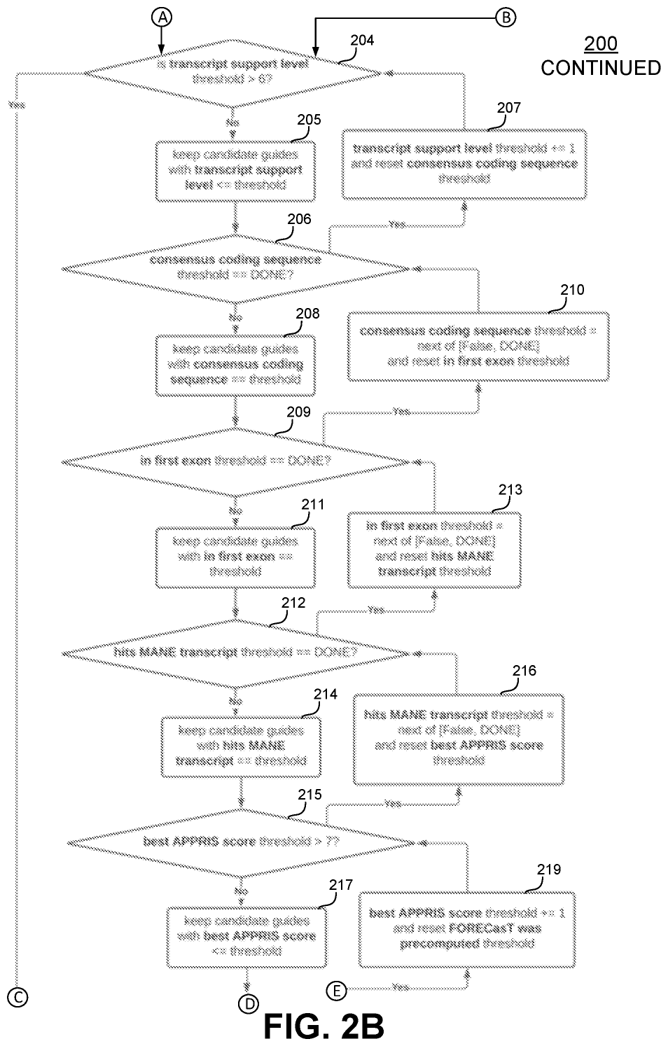

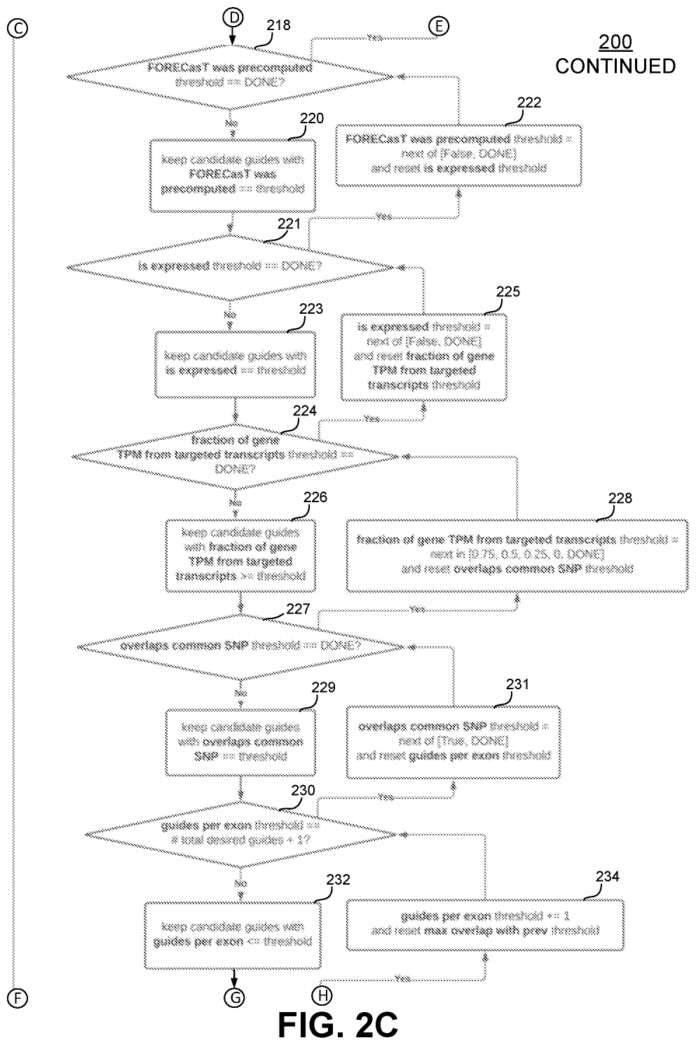

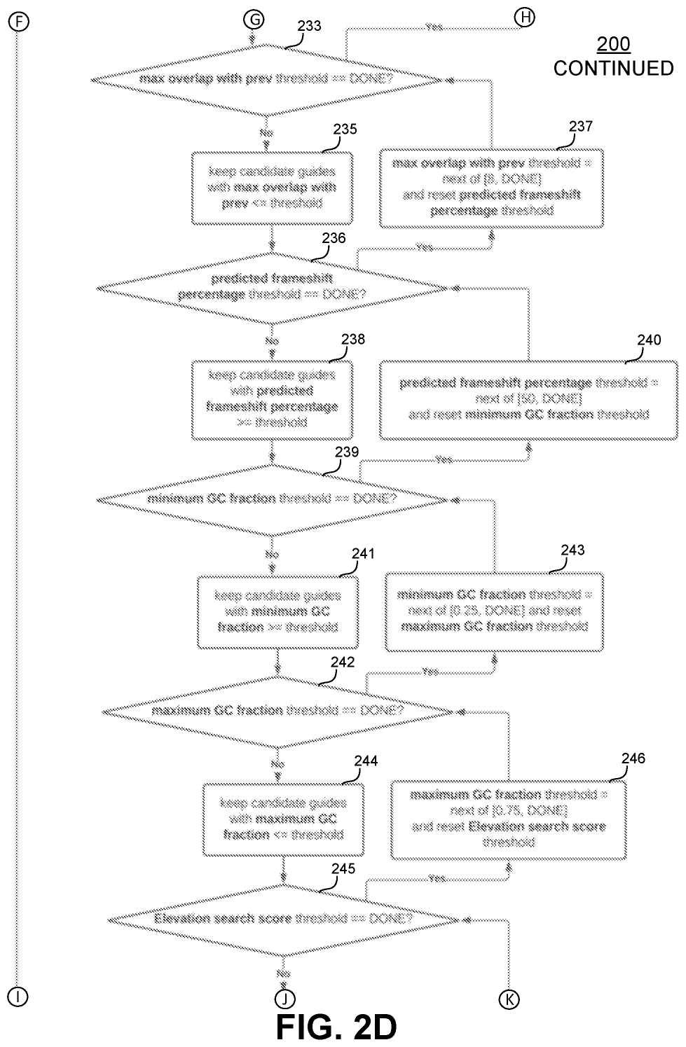

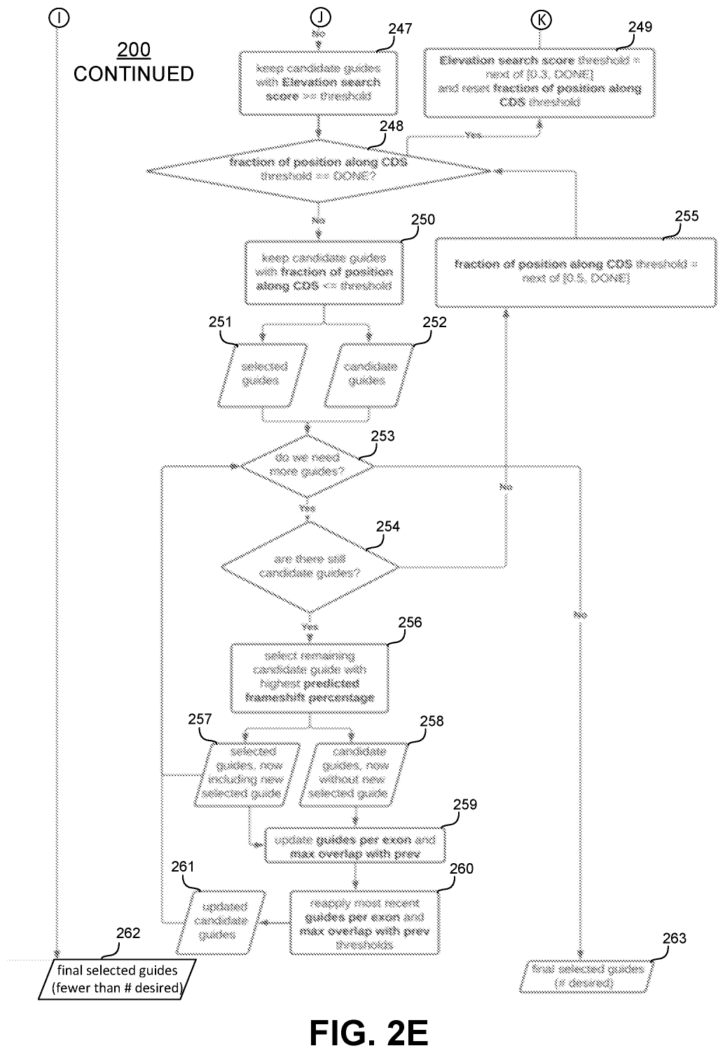

Transcript support level (TSL), initialized with a threshold at 201-3 of FIG. 2A, is a measure of assurance of the existence of a transcript. While some transcript models have a high level of support through the full length of their exon structure, there are also transcripts that are poorly supported and that should be considered speculative. TSL relies on primary data that can support full-length transcript structure. Ensembl employs the following TSL categories: TSL1—all splice junctions of the transcript are supported by at least one non-suspect mRNA; TSL2—the best supporting mRNA is flagged as suspect or the support is from multiple ESTs; TSL3—the only support is from a single EST; TSL4—the best supporting EST is flagged as suspect; TSL5—no single transcript supports the model structure; TSL-NA—the transcript was not analyzed for one of the following reasons: pseudogene annotation, including transcribed pseudogenes; human leukocyte antigen (HLA) transcript; immunoglobin gene transcript; T-cell receptor transcript; or single-exon transcript. One guide selection algorithm set forth herein employs the six TSL levels just described, initiating with TSL level 1. The last threshold to be relaxed is the TSL level: once TSL advances past TSL level 6, the guide selection process is terminated.

In certain embodiments, it may be preferable to avoid stringent guide selection on the basis of TSL. In certain embodiments, it may be advantageous for the guide selection algorithm to initiate at a TSL greater than one as an initialization value 201-3. For example, there may be coding sequences which have RefSeq and/or CCDS transcripts but none of the principal isoforms meet the stringency of TSL 1, 2 or 3.

Matched annotation between NCBI and EBI (MANE) was established to produce a genome-wide transcript set for human genes. For a transcript to be designated as a MANE transcript it must perfectly align to GRCh38, have complete sequence identity with a corresponding RefSeq transcript and be high-confidence in terms of its overall support. The MANE transcript set includes one well-supported transcript per protein-coding locus. The MANE Plus Clinical set includes additional transcripts required to report variants of clinical interest that cannot be reported using the MANE Select set. When used in a guide selection procedure, MANE is a binary selection filter. That is, a target coding sequence either is or is not part of a MANE transcript. A threshold for MANE transcript may be binary and is set at 201-6.

APPRIS is a system to annotate alternatively spliced transcripts based on a range of computational methods. APPRIS attempts to select a single CDS variant for each gene as the main isoform, however this is not always possible. Principal isoforms are tagged with the numbers 1 to 5, with 1 being the most significant and if a principal variant cannot be chosen, a variant can be tagged with one of two alternative categories. The seven categories are reflected in the seven threshold levels of the guide selection algorithm exemplified herein. In certain embodiments, fewer than all seven thresholds are tested.

Ensembl employs the APPRIS tags: PRINCIPAL:1—Transcript(s) expected to code for the main functional isoform based solely on the core modules in the APPRIS; PRINCIPAL:2 —Where the APPRIS core modules are unable to choose a clear principal variant (approximately 25% of human protein coding genes), the database chooses two or more of the CDS variants as “candidates” to be the principal variant. PRINCIPAL:3—Where the APPRIS core modules are unable to choose a clear principal variant and more than one of the variants have distinct consensus coding sequence (CCDS) identifiers, APPRIS selects the variant with lowest CCDS identifier as the principal variant. The lower the CCDS identifier, the earlier it was annotated. PRINCIPAL:4—Where the APPRIS core modules are unable to choose a clear principal CDS and there is more than one variant with a distinct (but consecutive) CCDS identifiers, APPRIS selects the longest CCDS isoform as the principal variant. PRINCIPAL:5—Where the APPRIS core modules are unable to choose a clear principal variant and none of the candidate variants are annotated by CCDS, APPRIS selects the longest of the candidate isoforms as the principal variant. In certain embodiments, thresholds correspond to APPRIS scores. A threshold for best APPRIS score is set at 201-7.

For genes in which the APPRIS core modules are unable to choose a clear principal variant (approximately 25% of human protein coding genes), the “candidate” variants not chosen as principal are labeled in the following way: ALTERNATIVE:1—Candidate transcript(s) models that are conserved in at least three tested species; ALTERNATIVE:2—Candidate transcript(s) models that appear to be conserved in fewer than three tested species.

GENCODE Basic provides another useful annotation source. GENCODE Basic is a subset of the GENCODE gene set, and is intended to provide a simplified, high-quality subset of the GENCODE transcript annotations. This subset prioritizes full-length protein coding transcripts over partial or non-protein coding transcripts within the same gene.

In the selection methods exemplified herein, consensus coding sequence 201-4 refers to a range of a coding sequence that is common among all protein-coding transcripts of a locus with a particular support level or better. For example, using TSL=3, a consensus coding sequence is present in all transcripts having TSL=3 or better.

In certain embodiments, first exons are dropped 201-5. Many mRNA transcripts are capable of translation from ATGs downstream from the first exon. Often, removing the first ATG or disrupting translation from the first ATG allows or enhances translation from an alternate ATG. Dropping the first coding exon eliminates candidate guides that target upstream of an alternative translation initiation site and increases the likelihood that a frameshift will be a knockout.

It is known that the mutational outcomes are not random but depend on DNA sequence at the targeted location. FORECasT is a computational predictor of the mutational outcomes of a given guide RNA. FORECasT provides prediction tools and precomputed profiles of all gRNAs in human and mouse coding regions. A threshold for FORECasT score is set at 201-8.

The guide selection procedure includes a binary filter 201-9 to avoid coding sequences for which an expression product has not yet been identified. The initiation threshold of the filter 201-9 asks that the coding sequence is expressed.

Transcripts per million (TPM) is a normalization method for RNA-seq to correct for transcript length. For certain guide selection methods, the initialization state of a filter 201-10 tests whether the TPM is greater than a threshold value. The filter for fraction of TPM from targeted transcripts can be binary (i.e., meeting a preset threshold is either true or false) or the threshold can be incremental (i.e., beginning with a stringent threshold and incrementally relaxed as selection progresses). In an exemplified guide selection procedure, in later iterations, the required TMP is relaxed to lower expression levels.

With continued reference to FIG. 2A, in certain embodiments, the initialization state 201 of the guide selection procedure 200 is to filter out 201-11 guides that overlap common single nucleotide polymorphisms (SNPs). Ensembl includes data sources to identify SNPs.

In certain embodiments, the guide selection procedure accounts for and limits the number of guides selected per exon. The initialization state 201-1 of the filter is to limit the number of guides per exon to 1. Once a first guide is selected, guides that bind to that exon are rejected while guides accumulate to other exons. The number of guides per exon threshold is initialized at 201-12, and is raised in later iterations.

In certain embodiments, the guide selection procedure accounts for and limits overlap of a candidate guide with any previously selected guide. In certain embodiments, the initialization state 201-2 of the guide overlap filter is to reject candidate guides that overlap to any degree (i.e., maximum overlap with a previous guide=0). In subsequent iterations, the filter 201-13 is relaxed to incrementally allow overlap by 1, 2, 3, 4, 5, 6, 7, or 8 nucleotides.

Elevation is an approach to prediction of off-target effects in CRISPR systems. Elevation includes pre-computed on-target and off-target activity prediction or the human genome. A threshold for elevation search score may be initialized at 201-17.

The method further comprises a selection filter 201-14 for predicted frameshift percentage, which may be established from target data. In certain embodiments, the target data can be from a subset of cells or tissue types. In certain embodiments, the target data is representative of a cell or tissue type. In certain embodiments, the target data is representative of a disease state. Typically, a frameshift prediction filter is trained on frameshifts observed in cells following transfection (e.g., nucleofection or lipofection) of unique guides. For example, the inventors produced frameshift models using unique guides to target multiple genes in human umbilical vascular endothelial cells (HUVEC), retinal pigmented epithelium cells (ARPE-19); and other cell types.

The model employs a predicted frameshift percentage threshold 201-14. Frameshift percentage refers to the percentage of sequenced amplicons that have a frameshift-inducing mutation after a given guide sequence is used in a population of cells. In certain embodiments, a frameshift percentage prediction model is employed. An exemplary frameshift percentage prediction model features without limitation, one or more of, or a combination of: 1) one-hot encoding of bases at guide target sequence positions from −4 to +26, 2) location of the guide on the cDNA (bp from start, bp from end, fraction from start, each as an average over transcripts weighted by expression), 3) location of the guide on the CDS (bp from start, bp from end, fraction from start, each as an average over transcripts weighted by expression), 4) GC fraction of the target sequence, 5) expression of transcripts containing the target sequence in the target cell type, 6) expression of the targeted gene in the target cell type, 7) epigenetic features of the targeted gene in the target cell type including i) DNase sensitivity (broad, narrow; associated with chromatin remodeling and accessibility to transcription factors), ii) histone H3 lysine 4 trimethylation (H3K4me3) associated with gene activation, and iii) 7 epigenetic states inferred by merging ChromHMM and Segway segmentations, 8) binary overlap with common SNPs, 9) predicted fraction of in-frame mutations from FORECasT, 10) Azimuth on-target score, 11) DeepCRISPR on-target score, and 12) VBC score. The 7 epigenetic states of the ChromHMM/Segway segmentations characterize regions of a gene as CTCF enriched elements; predicted enhancers (E), predicted promoter flanking regions (PF), predicted repressed or low activity regions (R), predicted transcribed regions (T), predicted promoter regions (TSS), and predicted weak enhancer or open chromatin cis regulatory elements (WE). The resulting model is used to predict frameshift percentage for candidate guides. In the guide selection procedure of Example 2, the candidate guide with the highest predicted frameshift percentage is chose from the candidate guides that meet all of the selection criteria. A threshold for the fraction of position long CDS may be initialized at 201-18.

In certain embodiments, the frameshift percentage prediction model comprises an elastic net regression tuned by 10-fold cross-validation. The target variable is the mean frameshift percentage for each (target sequence, cell type) combination, as measured by Sanger sequencing of cells from nucleofection and lipofection experiments. Features the model uses for prediction include i) one-hot encoding of base at each zero-indexed position from −4 to 26, inclusive, but excluding 22 and 23 (the GG of the PAM sequence); ii) number and fraction of base pairs from the start and end of the cDNA and CDS (averaged across transcripts weighted by their expression); iii) the fraction of bases in the 20 bp target sequence that are G or C; iv) expression in primary HUVEC (mean RNAseq from ˜30 distinct cell batches, in transcripts per million), both the sum of expression across targeted transcripts and the total expression for the targeted gene, including non-targeted transcripts; iv) epigenetic data in HUVEC (public data), including binary overlap with DNAse broad and narrow peaks, h3k4me3 histone methylation narrow peaks (narrow), one-hot encoding of containing sequence type according to a combined ChromHMM/Segway 7-state model; v) binary overlap with common SNPs from dbSNP (above 0.01% in any of the 26 major populations used, as described below, vi) scores from third-party tools FORECasT (expected percentage of in-frame mutations), Azimuth on-target score, and DeepCRISPR on-target score. Example 2 employs such a frameshift prediction model.

The criteria used to consider whether SNP variation is common include 1) a variant has germline origin; 2) the variant has a minor allele frequency (MAF) of >=0.01 in at least one major population, with at least two unrelated individuals having the minor allele, and 3) MAF was computed with founder genotypes only. That is, if a variant's minor allele was observed only in a parent and its child, the variant is not considered “common”.

Accordingly, criteria for guide selection include, without limitation, one or more of transcript support level; targeting of a consensus coding sequence; targeting a coding sequence not within the first coding exon, targeting a MANE transcript; targeting a principal transcript (e.g., a transcript with a low APPRIS score), whether there is a precomputed prediction of editing outcomes (e.g., FORECasT), whether the coding sequence is observed to be expressed, the fraction of gene expression attributable to transcripts comprising the targeted coding sequence, whether there is overlap with a common sequence polymorphism (e.g., a SNP), limiting the number of guides selected for an exon, minimizing overlap with other guides that target an exon, the predicted or measured rate at which a guide induces a frameshift mutation, a GC fraction greater than a selected threshold, a GC fraction less than a selected threshold, low off-target activity, and position along a coding sequence. A minimum GC fraction threshold may be initialized at 201-15, while a maximum GC fraction threshold may be initialized at 201-17.

As set forth, the criteria can be applied in a binary manner (e.g., true or false), or over a range of values. The following non-limiting list of guide selection criteria includes in brackets more stringent “initialization” values applied at the start of a guide selection process, followed by less stringent “relaxed” values suitable to be applied later in the guide selectin process, for example when additional guides are desired. The numeric values are exemplary and different initialization values and relaxation ranges may be selected when suitable. Example 2, described below, employs all of the criteria in the following order relaxing the thresholds iteratively as depicted in FIG. 2. With reference to initialization 201 FIG. 2A, Table 1 shows the possible initialization states 201 (e.g., ranges or possible settings) of various filter aspects, with an example of actual initialization states illustrated in FIG. 2A.

TABLE 1
Initialization State Ranges

| Item | |
|---|---|
| Number | Initialization State |
| 201-3 | transcript support level 1, 2, 3, 4, 5, 6 (lower is more stringent) |
| 201-4 | consensus_coding_sequence [True, False] |
| 201-5 | drop_first_coding_exon [True, False] |
| 201-6 | Hits a MANE transcript [True, False] |
| 201-7 | Best APPRIS score [1, 2, 3, 4, 5, 6, 7] (i.e., principal 1-5, |
| | alternative 1-2) |
| 201-8 | FORECasT was precomputed [True, False] |
| 201-9 | is expressed [True, False] |
| 201-10 | Fraction of gene expression attributable to targeted transcripts ≥ |
| | [0.95, 0.75, 0.5, 0.25, 0] |
| 201-11 | Overlaps common SNP [False, True] |
| 201-12 | max_guides_per_exon [1, 2, 3, 4, 5, 6] |
| 201-13 | Max bp overlap with any previously selected guide ≤ [0, 8] |
| 201-14 | Predicted frameshift frac ≥ [.75, .5] |
| 201-15 | GC fraction ≥ [0.3, 0.25] |
| 201-16 | GC fraction ≤ [0.7, 0.75] |
| 201-17 | Elevation-search off-target score ≥ [.5, .3] (higher is better) |
| 201-18 | Position along CDS (fraction) ≤ [0.25, 0.5] |

Optionally, the criteria can be tested in a different order and/or using different thresholds. It will be appreciated that in certain embodiments, fewer that all of the criteria will be satisfied. In certain embodiments, one or more criteria can have relaxed starting thresholds that impose no selection. Moreover, in an iterative selection process, certain selection criteria will be relaxed to the point that no selection is imposed.

### Example Comparisons of Guides Selected Using the Described Technology

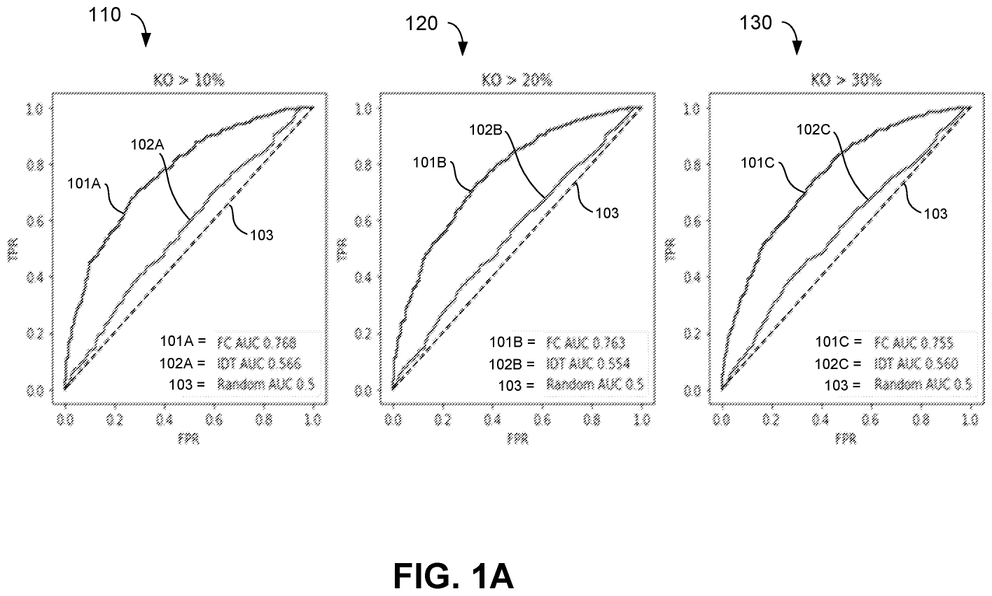

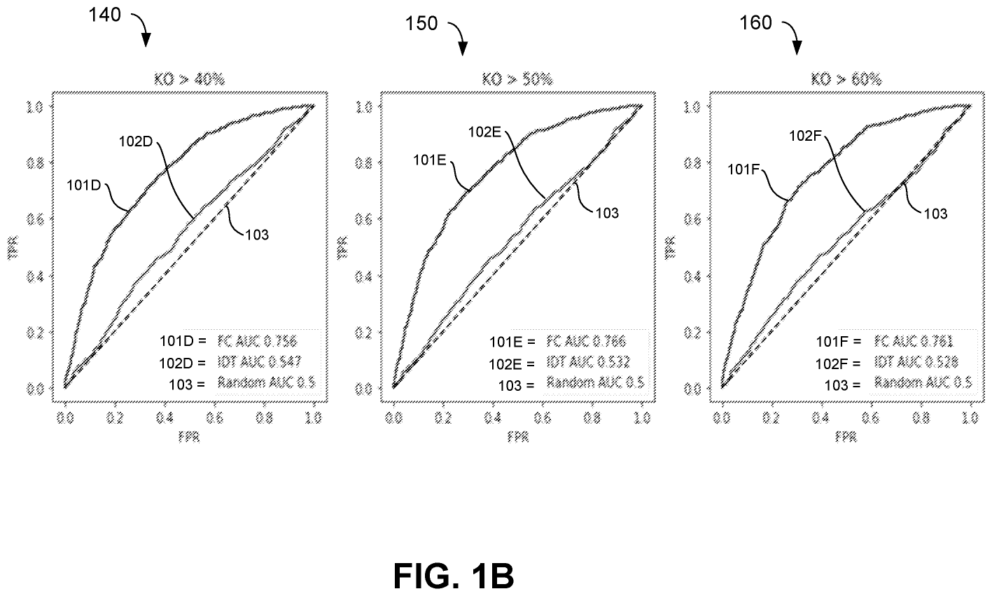

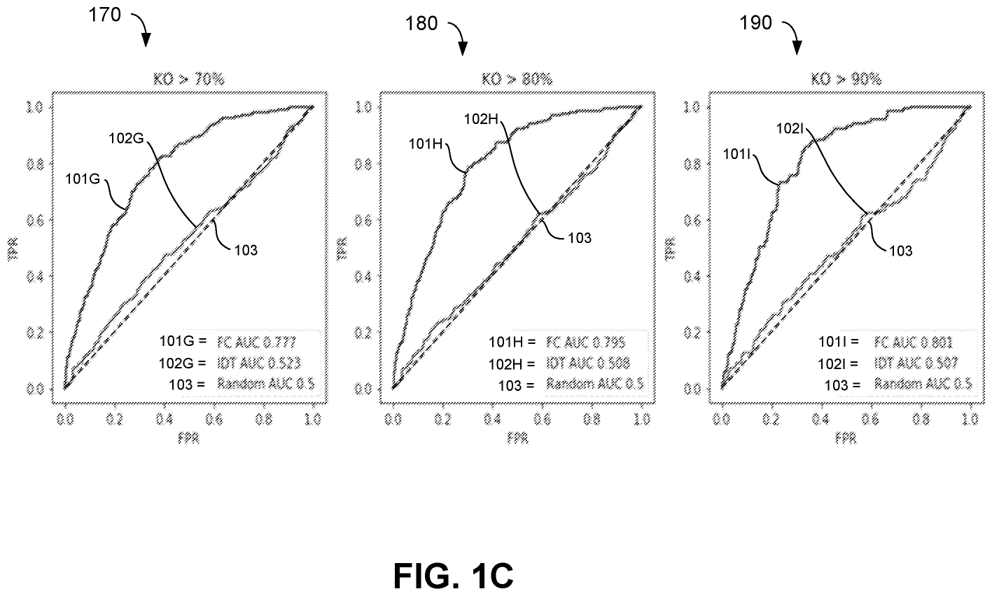

FIGS. 1A-1C depict head-to-head guide design comparisons of a plurality of guides selected according to embodiments described herein.

For example, in various embodiments, prospective guides may be compared to an Integrated DNA Technologies (IDT) design tool. TPR is the true positive rate and FPR is the false positive rate. Positive=a guide had a measured frameshift percentage above the threshold in question (10% in graph 110, then 20% in graph 120, and so on to 90% in graph 190). Frameshift percentage refers to the percentage of the sequenced amplicons that have a frameshift-inducing mutation after a given guide sequence is used in a population of cells. For the prediction model Frameshift percentage model (“FC”) curves, guides are ranked by their predicted (not measured) frameshift percentage, and the plot shows how TPR and FPR change as one proceeds down the ranked list, as well as the area under the curve. The “IDT” curves are derived the same way except that the guides are ranked by their IDT on-target score. Each graph 110-190 illustrates a comparison of the Area Under the Curve (AUC) its respective FC curve 101, its respective IDT curve 102, and a random curve (103) which has a slope of 1.

In FIGS. 1A-1C, Frameshift percentage model curves 101 are illustrated in comparison to curve from an IDT curve 102 and in further comparison comparison to a random curve 103 with a slope of 1. On each of the graphs, 110-190, the area under the curve (AUC) for each of the frameshift percentage model curve 101, the IDT design tool curve 102 and the random curve 103 are specified.

With reference to FIG. 1A, graph 110 illustrates a knock out (KO) score of greater than 10% and compares a frameshift model curve 101A, an IDT design tool curve 102A, and a random curve 103. The AUC for each is displayed in the lower right corner of graph 110. Graph 120 illustrates a knock out (KO) score of greater than 20% and compares a frameshift model curve 101B, an IDT design tool curve 102B, and the random curve 103. The AUC for each is displayed in the lower right corner of graph 120. Graph 130 illustrates a knock out (KO) score of greater than 30% and compares a frameshift model curve 101C, an IDT design tool curve 102C, and the random curve 103. The AUC for each is displayed in the lower right corner of graph 130.

With reference to FIG. 1B, graph 140 illustrates a knock out (KO) score of greater than 40% and compares a frameshift model curve 101D, an IDT design tool curve 102D, and the random curve 103. The AUC for each is displayed in the lower right corner of graph 140. Graph 150 illustrates a knock out (KO) score of greater than 50% and compares a frameshift model curve 101E, an IDT design tool curve 102E, and the random curve 103. The AUC for each is displayed in the lower right corner of graph 150. Graph 160 illustrates a knock out (KO) score of greater than 60% and compares a frameshift model curve 101F, an IDT design tool curve 102F, and the random curve 103. The AUC for each is displayed in the lower right corner of graph 160.

With reference to FIG. 1C, graph 170 illustrates a knock out (KO) score of greater than 70% and compares a frameshift model curve 101G, an IDT design tool curve 102G, and the random curve 103. The AUC for each is displayed in the lower right corner of graph 170. Graph 180 illustrates a knock out (KO) score of greater than 80% and compares a frameshift model curve 101H, an IDT design tool curve 102H, and the random curve 103. The AUC for each is displayed in the lower right corner of graph 180. Graph 190 illustrates a knock out (KO) score of greater than 90% and compares a frameshift model curve 101I, an IDT design tool curve 102I, and the random curve 103. The AUC for each is displayed in the lower right corner of graph 190.

### Recursive Procedure for CRISPR Guide Selection

FIGS. 2A-2E depict some example aspects of a recursive procedure for CRISPR guide selection, according to various embodiments. With reference to FIG. 2A, a user may adjust initialization states 201 in the manner previously described, and may provide user input 202 in the form of candidate guides 202-1 from which selections are made, selected guides 202-2 (which starts empty), and a desired number of guides 202-3 to be selected. Examples of the process illustrated in FIGS. 2B-2E after initialization and user input in FIG. 2A are described below with respect to Examples 1, 2, and 3. With respect to FIGS. 2A-2E, a single process in illustrated across multiple Figures, connections between different portions of the process from Figure to Figure are represented by lettered circles (A through K). For example, Circled A on FIG. 2A, corresponds to circled A on FIG. 2B, the same protocol is applied to other circled letters

The process depicted in FIGS. 2B-2E implies a strict order of evaluation of attributes. For example, with the default attribute order, guides targeting transcripts with transcript support level (TSL) 1 are considered, relaxing all the other attributes before relaxing transcript support level to consider TSL 2. However, the user can change the order and thresholds of attributes if desired via initialization 201.

As used herein, the term “protospacer adjacent sequence” or “protospacer adjacent motif” or “PAM” refers to an approximately 2-6 base pair DNA sequence (or a 2-, 3-, 4-, 5-, 6-, 7-, 8-, 9-, 10-, 11-, 12-long nucleotide sequence) that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease may be modified to alter the PAM specificity of the nuclease such that the nuclease recognizes alternative PAM sequence.

For example, with reference to the canonical SpCas9 amino acid sequence, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These examples are not meant to be limiting. It will be further appreciated that non-SpCas9s bind a variety of PAM sequences, which makes them useful to expand the range of target sequences that can be knocked out according to the various embodiments. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

The guide molecule or guide RNA of a Class 2 type V CRISPR-Cas protein comprises a tracr-mate sequence (encompassing a “direct repeat” in the context of an endogenous CRISPR system) and a guide sequence (also referred to as a “spacer” in the context of an endogenous CRISPR system).

In general, a CRISPR system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

The terms “guide molecule” and “guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

As used herein, the term “crRNA” or “guide RNA” or “single guide RNA” or “sgRNA” or “one or more nucleic acid components” of a Type V or Type VI CRISPR-Cas locus effector protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign, ELAND (Illumina, San Diego, Calif.), SOAP, and Maq. The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA and pre-mRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree of secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold. Another example folding algorithm is the online webserver RNAfold, developed at Institute for Theoretical Chemistry at the University of Vienna, using the centroid structure prediction algorithm.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

### Guide Modifications

In certain embodiments, guides of comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment, a guide nucleic acid comprises ribonucleotides and non-ribonucleotides. In one such embodiment, a guide comprises one or more ribonucleotides and one or more deoxyribonucleotides. In an embodiment, the guide comprises one or more non-naturally occurring nucleotide or nucleotide analog such as a nucleotide with phosphorothioate linkage, boranophosphate linkage, a locked nucleic acid (LNA) nucleotide comprising a methylene bridge between the 2′ and 4′ carbons of the ribose ring, peptide nucleic acids (PNA), or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), polyethylene glycol (PEG), triethylene glycol, or tetraethyleneglycol (TEG). Further examples of modified bases include, but are not limited to, 2-aminopurine, 5-bromo-uridine, pseudouridine, N1-methylpseudouridine, 5-methoxyuridine (5moU), inosine, and 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl (cEt), 2′-O-methyl-3′-thioPACE (MSP), or 2′-O-methyl-3′-phosphonoacetate (MP) at one or more terminal nucleotides. Such chemically modified guides can comprise increased stability and increased activity as compared to unmodified guides, though on-target vs. off-target specificity may not be predictable. In some embodiments, the 5′ and/or 3′ end of a guide RNA is modified by a variety of functional moieties including fluorescent dyes, polyethylene glycol, cholesterol, proteins, or detection tags. In an embodiment, deoxyribonucleotides and/or nucleotide analogs are incorporated in engineered guide structures, such as, without limitation, 5′ and/or 3′ end, stem-loop regions, and the seed region.

### Tandem Guides and Multiplexing

CRISPR enzymes can employ more than one RNA guide without losing activity. This enables the use of the CRISPR enzymes, systems or complexes as defined herein for targeting multiple DNA targets, genes or gene loci, with a single enzyme, system or complex as defined herein. The guide RNAs may be tandemly arranged, optionally separated by a nucleotide sequence such as a direct repeat as defined herein. The position of the different guide RNAs in the tandem does not influence the activity.

Accordingly, the CRISPR enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional CRISPR system or complex binds to the multiple target sequences. In some embodiments, the functional CRISPR system or complex may edit the multiple target sequences. Examples of multiplex genome engineering using CRISPR effector proteins are provided in Cong et al. and other publications cited herein. More specifically, multiplex gene editing using Cpf1 is well known to those of ordinary skill in the arts.

Any of the methods, products, compositions and uses as described herein elsewhere are equally applicable with the multiplex or tandem targeting approach further detailed below. By means of further guidance, the following particular aspects and embodiments are provided.

### Delivery Methods

Some aspects comprise methods for delivering one or more polynucleotides, such as one or more vectors encoding one or more components described herein, one or more transcripts thereof, and/or one or proteins transcribed therefrom, to a host cell. In some aspects, the described technology provides cells produced by such methods, and organisms (such as animals, plants, or fungi) comprising or produced from such cells. In some embodiments, a CRISPR Cas9 as described herein in combination with (and optionally complexed with) a guide sequence is delivered to a cell.

Conventional viral and non-viral based gene transfer methods can be used to introduce nucleic acids in mammalian cells or target tissues. Such methods can be used to administer nucleic acids encoding components of a genome editor to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

Methods of non-viral delivery of nucleic acids include lipofection, nucleofection, microinjection, biolistics, virosomes, liposomes, immunoliposomes, polycation or lipidmucleic acid conjugates, naked DNA, artificial virions, and agent-enhanced uptake of DNA. Lipofection is well known to those of ordinary skill in the arts and lipofection reagents are sold commercially (e.g., Transfectam™ and Lipofectin™).

Many cationic and neutral lipids are suitable for efficient receptor-recognition lipofection of polynucleotides. Delivery can be to cells (e.g., in vitro or ex vivo administration) or target tissues (e.g., in vivo administration).

The preparation of lipid-nucleic acid complexes, including targeted liposomes such as immunolipid complexes, is well known to one of skill in the art.

### Additional CRISPR-Cas Development and Use Considerations

Various embodiments of the described technology may be further illustrated and extended based on aspects of CRISPR-Cas9 development and use as set forth in the following articles and particularly as relates to delivery of a CRISPR protein complex and uses of an RNA guided endonuclease in cells and organisms.

With respect to general information on CRISPR-Cas Systems, components thereof, and delivery of such components, including methods, materials, delivery vehicles, vectors, particles, AAV, and making and using thereof, including as to amounts and formulations, all useful in the practice of the described technology.

The technology described herein may be used as part of a research program wherein there is transmission of results or data. A computer system (or digital device) may be used to receive, transmit, display and/or store results, analyze the data and/or results, and/or produce a report of the results and/or data and/or analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to various embodiments can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers). In some embodiments, the computer system comprises one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in various embodiments. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users. A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. Accordingly, various embodiments of the described technology comprehend performing any method herein-discussed and storing and/or transmitting data and/or results therefrom and/or analysis thereof, as well as products from performing any method herein-discussed, including intermediates.

### Library Generation Architectures

An operating environment includes networked devices that are configured to communicate with one another via one or more networks. In some embodiments, the user device or genomic sequence input device may provide nucleic acid sequence data directly from user input, or from sequencing data, such as obtained from sequencing. In other example embodiments, the genomic sequence input device may be obtained from a remote server comprising said sequence information. In some embodiments, a user associated with a device must install an application and/or make a feature selection to obtain the benefits of the techniques described herein. The guide sequence library selection system receives sequence data and sequence annotation data and outputs a set of identified ranked guide sequence. The ranking of individual guide sequence reflects the likelihood of a given guide sequence being able to recognize, hybridize or bind to, and/or induce a frameshift mutation in a cell of an organism such as a mammal or a human or a mouse.

Each network device includes a device having a communication module capable of transmitting and receiving data over the network. For example, each network device can include a server, desktop computer, laptop computer, tablet computer, a television with one or more processors embedded therein and/or coupled thereto, smart phone, handheld computer, personal digital assistant (“PDA”), or any other wired or wireless, processor-driven device.

The genomic sequence input device may generate nucleic acid sequence data files comprising information on the coding regions or exons, or genes, within a given biological sample. In one example embodiment, the genomic sequence information input device may directly communicate the data file to the guide sequence library generation system across the network and the guide sequence library generation and ranking is conducted in line with the sequence input and/or analysis. In another example embodiment, the sequence information data file may be stored on a data storage medium and later uploaded to the guide sequence library generation system for further analysis.

The guide sequence library generation system may comprise an input module, an exon prediction module, a ranking module, and a graphical user interface (GUI) module. The input module receives input data from genomic sequence information input device and formats such data for further processing. The exon prediction module takes the genomic input information and identifies exon sequences in order to identify an initial set of target regions. The ranking module takes the identified target regions and generates a set of ranked guide sequences for each target region. The output module then formats and displays this information to an end user. In certain example embodiments an output module may be configured through GUI to allow direct user interaction with guide library, for example by selecting a final set of guides or modifying certain input parameters to further refine the final guide sequence library produced. The guide sequence generation system may further optionally comprise a guide sequence index where guide sequence libraries are stored during and after guide sequence library production.

It will be appreciated that the network connections indicated are examples and other means of establishing a communications link between the computers and devices can be used. Moreover, those having ordinary skill in the art having the benefit of the present disclosure will appreciate that the guide selection system, can have any of several other suitable computer system configurations.

The present technology will be further illustrated in the following Examples which are given for illustration purposes only and are not intended to limit the described embodiments in any way.

### Example Processes

### Example 1

With reference again to FIGS. 2A-2E, an iterative model 200 for CRISPR guide selection is described. Initialization 201 and user input 202 of FIG. 2A, have previously been described. Turning to FIG. 2B, starting from initiation threshold levels 204, candidate guides are kept as candidates if they meet: the thresholds for transcript support level at (204, 205); consensus coding sequence (206, 208, with iteration at 207); first coding exon (209, 211, with iteration at 210); MANE threshold satisfied (212, 214, with iteration at 213); and APPRIS score threshold exceeded (215, 217 with iteration at 216). In the case that the transcript support level checked at 204 is greater than the set threshold, the iterative model moves to 262 of FIG. 2E, where it is noted that the final number of selected guides is fewer than the desired number specified by user input 202-3.

Continuing the process with reference to both FIGS. 2B and 2C, candidate guides are kept as candidates if they satisfy: a threshold for FORECast (218, 220, with iteration at 219); an expressed threshold is satisfied (221, 223, with iteration at 222); a fraction of gene TPM from targeted transcripts threshold (224, 226, with iteration at 225); overlaps a common SNP (227, 229, with iteration at 228); and maximum guides per exon (230, 232, with iteration at 231)

Continuing the process with reference to FIGS. 2C, 2D, and 2E, candidate guides are kept as candidates if they satisfy: a threshold for maximum overlap with previous threshold 233, 235, with iteration at 234); predicted frameshift percentage (236, 238, with iteration at 237); minimum GC fraction (239, 241, with iteration at 240), maximum GC fraction (242, 244, with iteration at 243); and elevation threshold score (245, 247, with iteration at 246).

Continuing the process with reference to FIGS. 2C, 2D, and 2E, candidate guides are kept as candidates if they satisfy: position along a coding sequence (248, 250, with iteration at 249); off-target score; fraction of expression due to targeted transcript; maximum overlap to a previously selected guide; and predicted knockout score.

With reference again to Table 1, initial thresholds are the first of the bracketed values. The candidate guide of candidate guides 252 with the highest knockout score is selected, stored values of selected guides updated 251 (e.g., guides per exon and location of selected guides used in determining whether the maximum overlap threshold is met) and the procedure is repeated. It is determined if there are still candidate guides to process (254), if not, the process moves to 255 where a fraction of the position along the CDS threshold is adjusted. If yes, the process moves to 256 where a remaining candidate guide with the highest predicted frameshift percentage is selected, removed from the candidate guides (257, 258) and the question is then asked at 253 as to whether more candidate guides are needed to achieve results requested by user input 202. at 258 the candidate and selected guides are updated per exon and max overlap; at 260 the most recent guides are reapplied per exon an max overlap with previous thresholds, and the list of candidate guides is revised 261 and the question is again asked as to whether more candidate guides are needed to achieve results requested by user input 202.

With respect to the iteration procedures in FIGS. 2B-2E, when there are no guide candidates remaining that satisfy the selection, the predicted % knockout threshold is relaxed from 75% and the procedure is repeated. For each successive stage of the selection, when there are no more guide candidates that satisfy that stage of the selection, the threshold of that selection is relaxed, and the selection is repeated. Relaxation of a threshold is usually accompanied by resetting thresholds of selections lower in the hierarchy to their initialization thresholds or to a less relaxed threshold. For example, a binary threshold is reset to its True or False initialization value. For a threshold having incremental values, the threshold is reset to a higher stringency value or the initialization value. The process is finished when a desired at 253 no more guides are needed because the final number of candidate guides selected a has achieved the desired number supplied as user input 202-3.

### Example 2

The iterative selection model illustrated in FIGS. 2A-2E is followed in a similar fashion with respect to Example 2. Starting from initiation threshold levels and user input described on FIG. 2A, candidate guides are kept as candidates if they meet the thresholds for transcript support level (TSL), consensus coding sequence, first exon, MANE transcript, APPRIS score, FORECasT is precomputed, fraction of gene TPM from targeted transcripts, overlaps common SNP, guides per exon, maximum overlap with a previously selected guide target, predicted frameshift percentage (or predicted knockout percentage), minimum GC fraction, maximum GC fraction, off target elevation search score, and fraction of position along the coding sequence. Initial thresholds are the first of the bracketed values (see, e.g., Table 1).

The candidate guide with the highest knockout score is added to the list of final selected guides, stored values of selected guides are updated (e.g., guides per exon and location of selected guides used in determining whether the maximum overlap threshold is met are stored) and the procedure is repeated. When there are no guide candidates remaining that satisfy all of the selection thresholds, the threshold for the fraction of position along a coding sequence is incrementally relaxed, and the procedure is repeated. For each iteration of the selection, when there are no more guide candidates that satisfy that stage of the selection, the threshold of that selection is relaxed, and the selection is repeated. Relaxation of a threshold is usually accompanied by resetting the threshold of one or more selections lower in the hierarchy to a higher stringency which can be the initialization threshold. For example, a binary threshold would be reset to its True or False initialization value. For a threshold having incremental values, the threshold would be reset to higher stringency value or the initialization value. The process is completed when a desired number of candidate guides has been selected.

### Example 3

Starting from guides mapping to 173 genes for which there is frameshift prediction data, for each gene in turn, a frameshift percentage prediction model described herein was trained on the guides for all the other genes, and frameshift percentage was predicted for the guides of the held-out gene. For 9 threshold percentages (10, 20 . . . 90), the measured frameshift percentage was binarized and the area under the curve (AUC) was evaluated for the model's predicted frameshift percentage compared to an Integrated DNA Technologies (IDT) design tool on-target score. Results are depicted in FIGS. 1A-1C. TPR is the true positive rate and FPR is the false positive rate. Positive=a guide had a measured frameshift percentage above the threshold in question (10% in graph 110, then 20% in graph 120, and so on to 90% in graph 190). Frameshift percentage refers to the percentage of the sequenced amplicons that have a frameshift-inducing mutation after a given guide sequence is used in a population of cells. For the predicted frameshift percentage model (“FC”) curves (e.g., the curves illustrated in FIGS. 1A-1C), guides are ranked by their predicted (not measured) frameshift percentage, and the plot shows how TPR and FPR change as one proceeds down the ranked list, as well as the area under the curve. The “IDT” curves are derived the same way except that the guides are ranked by their IDT on-target score.

### Example Computer System Environment

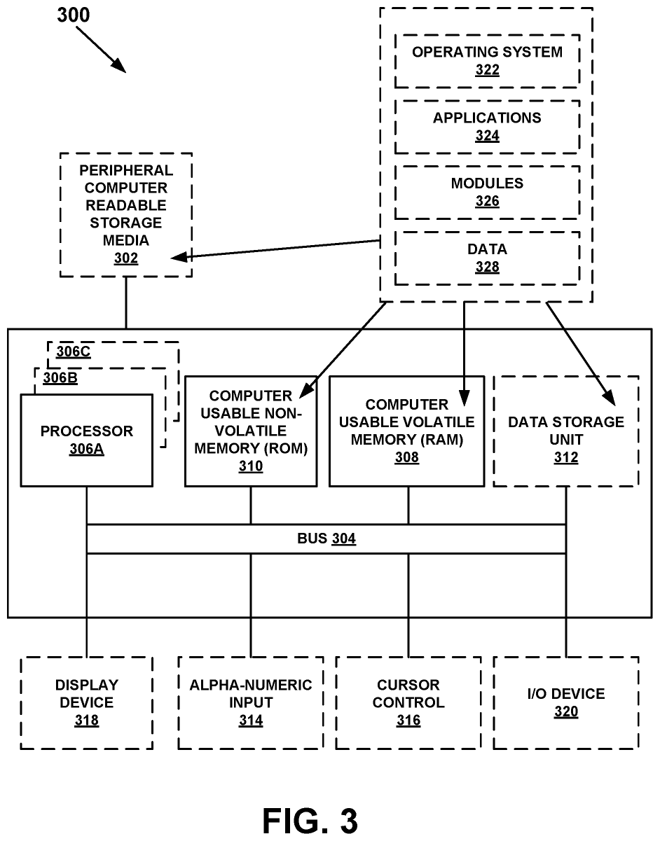

FIG. 3 illustrates components of an example computer system 300, with which or upon which, various embodiments may be implemented. With reference now to FIG. 3, all or portions of some embodiments described herein are composed of computer-readable and computer-executable instructions that reside, for example, in computer-usable/computer-readable storage media of a computer system. That is, FIG. 3 illustrates one example of a type of computer system 300 that can be used in accordance with or to implement various embodiments which are discussed herein. It is appreciated that computer system 300 of FIG. 3 is only an example and that embodiments as described herein can operate on or within a number of different computer systems including, but not limited to, general purpose networked computer systems, embedded computer systems, routers, switches, server devices, client devices, various intermediate devices/nodes, stand-alone computer systems, media centers, handheld computer systems, multi-media devices, and the like.

System 300 of FIG. 3 includes an address/data bus 304 for communicating information, and a processor 306A coupled with bus 304 for processing information and instructions. As depicted in FIG. 3, system 300 is also well suited to a multi-processor environment in which a plurality of processors 306A, 306B, and 306C are present. Conversely, system 300 is also well suited to having a single processor such as, for example, processor 306A. Processors 306A, 306B, and 306C may be any of various types of microprocessors. Computer system 300 also includes data storage features such as a computer usable volatile memory 308, e.g., random access memory (RAM), coupled with bus 304 for storing information and instructions for processors 306A, 306B, and 306C. System 300 also includes computer usable non-volatile memory 310, e.g., read only memory (ROM), coupled with bus 304 for storing static information and instructions for processors 306A, 306B, and 306C.

In some embodiments a data storage unit 312 (e.g., a magnetic or optical disk and disk drive) is coupled with bus 304 for storing information and instructions.

In some embodiments, computer system 300 is well adapted to having peripheral computer-readable storage media 302 such as, for example, a floppy disk, a compact disc, digital versatile disc, other disc-based storage, universal serial bus flash drive, removable memory card, and the like coupled thereto.

Computer system 300 may also include an optional alphanumeric input device 314 including alphanumeric and function keys coupled with bus 304 for communicating information and command selections to processor 306A or processors 306A, 306B, and 306C. Computer system 300 may also include an optional cursor control device 316 coupled with bus 304 for communicating user input information and command selections to processor 306A or processors 306A, 306B, and 306C. In some embodiments, system 300 also includes an optional display device 318 coupled with bus 304 for displaying information.

Optional cursor control device 316 allows the computer user to dynamically signal the movement of a visible symbol (cursor) on a display screen of display device 318 and indicate user selections of selectable items displayed on display device 318. Alternatively, it will be appreciated that a cursor can be directed and/or activated via input from optional alphanumeric input device 314 using special keys and key sequence commands. Computer system 300 is also well suited to having a cursor directed by other means such as, for example, voice commands.

In some embodiments, computer system 300 also includes an I/O device 320 for coupling system 300 with external entities. For example, in one embodiment, I/O device 320 is a modem for enabling wired or wireless communications between system 300 and an external device or network such as, but not limited to, the Internet.

Referring still to FIG. 3, various other components are depicted for system 300. Specifically, when present, an operating system 322, applications 324, modules 326, and data 328 are shown as typically residing in one or some combination of computer usable volatile memory 308 (e.g., RAM), computer usable non-volatile memory 310 (e.g., ROM), and data storage unit 312. In some embodiments, all or portions of various embodiments described herein are stored, for example, as an application 324 and/or module 326 in memory locations within RAM 308, computer-readable storage media within data storage unit 312, peripheral computer-readable storage media 302, and/or other computer-readable storage media.

### Example Methods of Operation

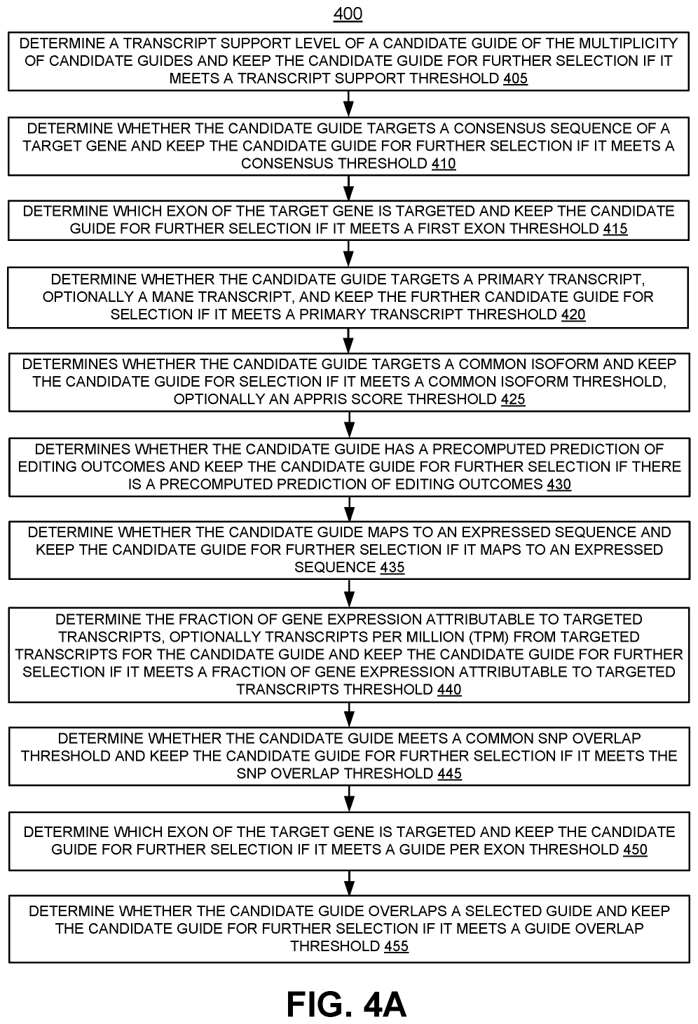

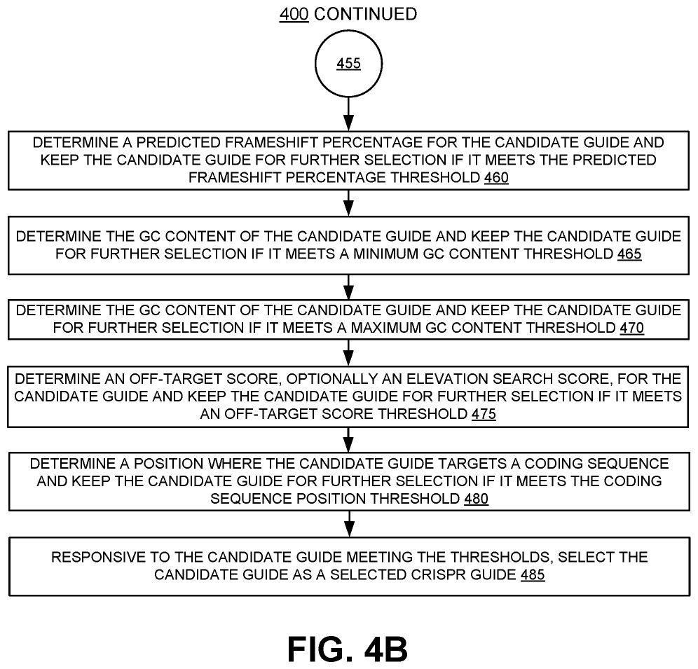

FIGS. 4A-4B illustrate a flow diagram 400 of an example a method of performing CRISPER guide selection, in accordance with various embodiments. More particularly, flow diagram 400 illustrates an example of a method of selecting CRISPR guides for knocking out one or more target genes in a target cell from a multiplicity of candidate guides. Procedures of the methods illustrated by flow diagram 400 of FIGS. 4A and 4B will be described with reference to aspects and/or components of one or more of FIGS. 1-4B. It is appreciated that in some embodiments, the procedures may be performed in a different order than described in a flow diagram, that some of the described procedures may not be performed, and/or that one or more additional procedures to those described may be performed. Flow diagram 400 include some procedures that, in various embodiments, are carried out by one or more processors or controllers (e.g., a processor 306, a controller 201, a computer 300, or the like) under the control of computer-readable and computer-executable instructions that are stored on non-transitory computer-readable storage media (e.g., peripheral computer-readable storage media 302, ROM 310, RAM 308, data storage unit 312, or the like). It is further appreciated that one or more procedures described in flow diagram 400 may be implemented in hardware, or a combination of hardware with firmware and/or software. The procedures illustrated in FIG. 4 may be implemented as an application 324 which may be stored upon and/or executed by computer 300.

With reference to FIG. 4A, at procedure 405 of flow diagram 400 in various embodiments, a processor such as processor 306, determines a transcript support level of a candidate guide of the multiplicity of candidate guides and keeping the candidate guide for further selection if it meets a transcript support threshold.

With continued reference to FIG. 4A, at procedure 410 of flow diagram 400, in various embodiments, a processor such as processor 306, determines whether the candidate guide targets a consensus sequence of a target gene and keeping the candidate guide for further selection if it meets a consensus threshold.

With continued reference to FIG. 4A, at procedure 415 of flow diagram 400, in various embodiments, a processor such as processor 306, determines which exon of the target gene is targeted and keeping the candidate guide for further selection if it meets a first exon threshold.

With continued reference to FIG. 4A, at procedure 420 of flow diagram 400, in various embodiments, a processor such as processor 306, determines whether the candidate guide targets a primary transcript, optionally a MANE transcript, and keeping the further candidate guide for selection if it meets a primary transcript threshold.

With continued reference to FIG. 4A, at procedure 425 of flow diagram 400, in various embodiments, a processor such as processor 306, determines whether the candidate guide targets a common isoform and keeping the candidate guide for selection if it meets a common isoform threshold, optionally an APPRIS score threshold.

With continued reference to FIG. 4A, at procedure 430 of flow diagram 400, in various embodiments, a processor such as processor 306, determines whether the candidate guide has a precomputed prediction of editing outcomes and keeping the candidate guide for further selection if there is a precomputed prediction of editing outcomes.

With continued reference to FIG. 4A, at procedure 435 of flow diagram 400, in various embodiments, a processor such as processor 306, determines whether the candidate guide maps to an expressed sequence and keeping the candidate guide for further selection if it maps to an expressed sequence.

With continued reference to FIG. 4A, at procedure 440 of flow diagram 400, in various embodiments, a processor such as processor 306, determines the fraction of gene expression attributable to targeted transcripts, optionally transcripts per million (TPM) from targeted transcripts for the candidate guide and keeping the candidate guide for further selection if it meets a fraction of gene expression attributable to targeted transcripts threshold.

With continued reference to FIG. 4A, at procedure 445 of flow diagram 400, in various embodiments, a processor such as processor 306, determines whether the candidate guide meets a common SNP overlap threshold and keeping the candidate guide for further selection if it meets the SNP overlap threshold.

With continued reference to FIG. 4A, at procedure 450 of flow diagram 400, in various embodiments, a processor such as processor 306, determines which exon of the target gene is targeted and keeping the candidate guide for further selection if it meets a guide per exon threshold.

With continued reference to FIG. 4A, at procedure 455 of flow diagram 400, in various embodiments, a processor such as processor 306, determines whether the candidate guide overlaps a selected guide and keeping the candidate guide for further selection if it meets a guide overlap threshold.

Referring now to FIG. 4B, at procedure 460 of flow diagram 400, in various embodiments, a processor such as processor 306, determines a predicted frameshift percentage for the candidate guide and keeping the candidate guide for further selection if it meets the predicted frameshift percentage threshold.

With continued reference to FIG. 4B, at procedure 465 of flow diagram 400, in various embodiments, a processor such as processor 306, determines the GC content of the candidate guide and keeping the candidate guide for further selection if it meets a minimum GC content threshold.

With continued reference to FIG. 4B, at procedure 470 of flow diagram 400, in various embodiments, a processor such as processor 306, determines the GC content of the candidate guide and keeping the candidate guide for further selection if it meets a maximum GC content threshold.

With continued reference to FIG. 4B, at procedure 475 of flow diagram 400, in various embodiments, a processor such as processor 306, determines an off-target score, optionally an elevation search score, for the candidate guide and keeping the candidate guide for further selection if it meets an off-target score threshold.

With continued reference to FIG. 4B, at procedure 480 of flow diagram 400, in various embodiments, a processor such as processor 306, determines a position where the candidate guide targets a coding sequence and keeping the candidate guide for further selection if it meets the coding sequence position threshold.

With continued reference to FIG. 4B, at procedure 485 of flow diagram 400, in various embodiments responsive to the candidate guide meeting the thresholds, a processor such as processor 306, selects the candidate guide as a selected CRISPR guide. This may comprise the processor selecting the candidate guide as the selected guide if it meets all of the thresholds and has the highest predicted frameshift percentage of the candidate guides.

In some embodiments of the method of flow diagram 400, the processor keeps the candidate guide for further selection if it meets a threshold for targeting a primary transcript or a main isoform of a transcript.

In some embodiments of the method of flow diagram 400, the processor rejects the candidate guide for further selection if it targets the first exon of a CDS.

In some embodiments of the method of flow diagram 400, the processor adjusts one or more thresholds and iterate the selection from the candidate guides until a desired number of selected guides are selected.

In some embodiments of the method of flow diagram 400, the candidate guides comprise guides of one of: a Type II CRISPR-Cas system, a Type V CRISPR-Cas system, and a Type VI CRISPR-Cas system.

In some embodiments of the method of flow diagram 400, the candidate guides comprise one of: an RNA guide; a DNA-RNA hybrid guide, or chemically modified bases guide.

In some embodiments of the method of flow diagram 400, the method may further comprise synthesizing the selected guide sequences.

### Alternative Embodiments

A method of selecting one or more CRISPR-Cas system guide sequences for generating loss-of-function mutations in coding sequences of target genes in a cell, which comprises: one or more steps to identify guide candidates that are biologically relevant, and one or more steps to identify candidate guides that optimally generate a functional knockout mutation.

The one or more steps to identify candidate guides for generating a functional knockout mutation comprises determining overlap of the candidate guide with polymorphisms of the target sequence, determining proximity of the candidate guide with epigenetic features of the target sequence, determining expression level of the target gene, identifying target genes and/or target sequences in a knock-out model, and/or determining or predicting targeting efficiency of the candidate guide.

The one or more steps to identify a guide candidate that is biologically relevant may comprise evaluating transcript support level of the target gene coding sequence, determining whether the target coding sequence is common to well supported protein coding transcripts of the target gene, determining whether the target sequence is in a first coding exon of a transcript of the target gene, determining whether the target sequence is present in a common isoform of a transcript of the target gene, and/or determining whether the target sequence is in a transcript designated to be the most prevalent transcript.

The one or more steps to identify a guide candidate may comprise discarding candidate guides that have multiple matches in the genome and/or discarding candidate guides predicted to have off-target effects.

The one or more steps to identify a guide candidate may comprise one or some combination of: i) identifying whether a candidate guide maps to a transcript; ii) identifying whether a candidate guide maps to a consensus coding sequence; iii) identifying whether a candidate guide maps to a translated exon; iv) identifying whether a candidate guide maps to a primary transcript; v) identifying whether a candidate guide maps to a main isoform; vi) identifying whether a candidate guide is predicted to introduce in-frame or frameshift mutations; vii) identifying whether a candidate guide maps to an expressed sequence; viii) identifying whether a candidate guide maps to a transcript that is expressed over a threshold level; ix) identifying whether a candidate guide overlaps a common SNP; x) identifying whether there are sufficient previously selected guides for an exon that the candidate guide maps to; xi) identifying to what extent a candidate guide overlaps with any previously selected guide; xii) identifying the fraction of mutations induced by a candidate guide predicted to be frameshift mutations; xiii) identifying whether the GC content of a candidate guide is above a lower limit; xiv) identifying whether the GC content of a candidate guide is below an upper limit; xv) identifying whether the off target activity of a candidate guide is predicted to be high; xvi) identifying whether the candidate guide maps to the N-terminal portion of the gene coding sequence; and/or xvii) selecting the guide if one or more of the conditions of (i) to (xvi) is satisfied. In some embodiments, the selectin may comprise finally selecting the guide if all of the conditions of (i) to (xvi) are satisfied and the guide has the highest predicted frameshift percentage of candidate guides that satisfy all of the conditions.

In some embodiments, one or more candidate guides are identified by adjacency to a PAM.

In some embodiments, the candidate guide is selected from a multiplicity of guides that target genes expressed in a particular cell type. In some embodiments, the cell type is a human umbilical vascular endothelial cell (HUVEC).

### CONCLUSION

The examples set forth herein were presented in order to best explain, to describe particular applications, and to thereby enable those skilled in the art to make and use embodiments of the described examples. However, those skilled in the art will recognize that the foregoing description and examples have been presented for the purposes of illustration and example only. The description as set forth is not intended to be exhaustive or to limit the embodiments to the precise form disclosed. Rather, the specific features and acts described above are disclosed as example forms of implementing the claims.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic the described technology.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular aspects, features, structures, or characteristics of any embodiment may be combined in any suitable manner with one or more other aspects, features, structures, or characteristics of one or more other embodiments without limitation.

What is claimed is:

1. A system for selecting CRISPR guides for knocking out one or more target genes in a target cell from a multiplicity of candidate guides, the system comprising:
a memory; and
a processor coupled with the memory and configured to:
determine a transcript support level of a candidate guide of the multiplicity of candidate guides and keeping the candidate guide for further selection if it meets a transcript support threshold;
determine whether the candidate guide targets a consensus sequence of a target gene and keeping the candidate guide for further selection if it meets a consensus threshold;
determine which exon of the target gene is targeted and keeping the candidate guide for further selection if it meets a first exon threshold;
determine whether the candidate guide targets a primary transcript and keeping the candidate guide for further selection if it meets a primary transcript threshold;
determine whether the candidate guide targets a common isoform and keeping the candidate guide for further selection if it meets a common isoform threshold;
determine whether the candidate guide has a precomputed prediction of editing outcomes and keeping the candidate guide for further selection if there is a precomputed prediction of editing outcomes;
determine whether the candidate guide maps to an expressed sequence and keeping the candidate guide for further selection if it maps to an expressed sequence;
determine a fraction of gene expression attributable to targeted transcripts for the candidate guide and keeping the candidate guide for further selection if it meets a fraction of gene expression attributable to targeted transcripts threshold;
determine whether the candidate guide meets a common SNP overlap threshold and keeping the candidate guide for further selection if it meets an SNP overlap threshold;
determine which exon of the target gene is targeted and keeping the candidate guide for further selection if it meets a guide per exon threshold;
determine whether the candidate guide overlaps a selected guide and keeping the candidate guide for further selection if it meets a guide overlap threshold;
determine a predicted frameshift percentage for the candidate guide and keeping the candidate guide for further selection if it meets a predicted frameshift percentage threshold;
determine a GC content of the candidate guide and keeping the candidate guide for further selection if it meets a minimum GC content threshold;
determine the GC content of the candidate guide and keeping the candidate guide for further selection if it meets a maximum GC content threshold;
determine an off-target score for the candidate guide and keeping the candidate guide for further selection if it meets an off-target score threshold;
determine a position where the candidate guide targets a coding sequence and keeping the candidate guide for further selection if it meets a coding sequence position threshold; and
responsive to the candidate guide meeting the thresholds, select the candidate guide as a selected CRISPR guide.

2. The system of claim 1, wherein the processor is further configured to select the candidate guide as the selected guide if it meets all the thresholds and has the highest predicted frameshift percentage of the candidate guides.

3. The system of claim 1, wherein the processor is further configured to keep the candidate guide for further selection if it meets a threshold for targeting a primary transcript or a main isoform of a transcript.

4. The system of claim 1, wherein the processor is further configured to reject the candidate guide for further selection if it targets a first exon of a CDS.

5. The system of claim 1, wherein the processor is further configured to adjust one or more thresholds and iterate the determination and the selection from the candidate guides until a desired number of selected guides are selected.

6. The system of claim 1, wherein the off-target score comprises an elevation search score.

7. The system of claim 1, wherein the candidate guides comprise guides of one of: a Type II CRISPR-Cas system, a Type V CRISPR-Cas system, and a Type VI CRISPR-Cas system.

8. The system of claim 1, wherein the candidate guides comprise one of: an RNA guide, a DNA-RNA hybrid guide, and a chemically modified base guide.

9. The system of claim 1, wherein the primary transcript a MANE transcript.

10. The system of claim 1, wherein the common isoform threshold comprises an APPRIS score threshold.

11. The system of claim 1, wherein the fraction of gene expression attributable to targeted transcripts comprises transcripts per million (TPM) from targeted transcripts for the candidate guide.

12. A method of selecting CRISPR guides for knocking out one or more target genes in a target cell from a multiplicity of candidate guides, the method comprising:
determining, by a processor, a transcript support level of a candidate guide of the multiplicity of candidate guides and keeping the candidate guide for further selection if it meets a transcript support threshold;
determining, by the processor, whether the candidate guide targets a consensus sequence of a target gene and keeping the candidate guide for further selection if it meets a consensus threshold;
determining, by the processor, which exon of the target gene is targeted and keeping the candidate guide for further selection if it meets a first exon threshold;
determining, by the processor, whether the candidate guide targets a primary transcript and keeping the candidate guide for further selection if it meets a primary transcript threshold;
determining, by the processor, whether the candidate guide targets a common isoform and keeping the candidate guide for further selection if it meets a common isoform threshold;
determining, by the processor, whether the candidate guide has a precomputed prediction of editing outcomes and keeping the candidate guide for further selection if there is a precomputed prediction of editing outcomes;
determining, by the processor, whether the candidate guide maps to an expressed sequence and keeping the candidate guide for further selection if it maps to an expressed sequence;
determining, by the processor, a fraction of gene expression attributable to targeted transcripts and keeping the candidate guide for further selection if it meets a fraction of gene expression attributable to targeted transcripts threshold;
determining, by the processor, whether the candidate guide meets a common SNP overlap threshold and keeping the candidate guide for further selection if it meets an SNP overlap threshold;
determining, by the processor, which exon of the target gene is targeted and keeping the candidate guide for further selection if it meets a guide per exon threshold;
determining, by the processor, whether the candidate guide overlaps a selected guide and keeping the candidate guide for further selection if it meets a guide overlap threshold;
determining, by the processor, a predicted frameshift percentage for the candidate guide and keeping the candidate guide for further selection if it meets a predicted frameshift percentage threshold;
determining, by the processor, a GC content of the candidate guide and keeping the candidate guide for further selection if it meets a minimum GC content threshold;
determining, by the processor, the GC content of the candidate guide and keeping the candidate guide for further selection if it meets a maximum GC content threshold;
determining, by the processor, an off-target score for the candidate guide and keeping the candidate guide for further selection if it meets an off-target score threshold;
determining, by the processor, a position where the candidate guide targets a coding sequence and keeping the candidate guide for further selection if it meets a coding sequence position threshold; and
responsive to the candidate guide meeting the thresholds, selecting, by the processor, the candidate guide as a selected CRISPR guide.

13. The method as recited in claim 12, further comprising: selecting, by the processor, the candidate guide as the selected guide if it meets all the thresholds and has the highest predicted frameshift percentage of the candidate guides.

14. The method as recited in claim 12, further comprising: keeping, by the processor, the candidate guide for further selection if it meets a threshold for targeting a primary transcript or a main isoform of a transcript.

15. The method as recited in claim 12, further comprising: rejecting, by the processor, the candidate guide for further selection if it targets a first exon of a CDS.

16. The method as recited in claim 12, further comprising: adjusting, by the processor, one or more thresholds and iterate the determination and the selection from the candidate guides until a desired number of selected guides are selected.

17. The method as recited in claim 12, wherein the off-target score comprises an elevation search score.

18. The method as recited in claim 12, wherein the candidate guides comprise guides of one of: a Type II CRISPR-Cas system, a Type V CRISPR-Cas system, and a Type VI CRISPR-Cas system.

19. The method as recited in claim 12, wherein the candidate guides comprise one of: an RNA guide, a DNA-RNA hybrid guide, and a chemically modified base guide.

20. A non-transitory computer readable storage medium comprising instructions embodied thereon, which when executed, cause a processor to perform a method of selecting CRISPR guides for knocking out one or more target genes in a target cell from a multiplicity of candidate guides, the method comprising:
determining, by a processor, a transcript support level of a candidate guide of the multiplicity of candidate guides and keeping the candidate guide for further selection if it meets a transcript support threshold;
determining, by the processor, whether the candidate guide targets a consensus sequence of a target gene and keeping the candidate guide for further selection if it meets a consensus threshold;
determining, by the processor, which exon of the target gene is targeted and keeping the candidate guide for further selection if it meets a first exon threshold;
determining, by the processor, whether the candidate guide targets a primary transcript and keeping the candidate guide for further selection if it meets a primary transcript threshold;
determining, by the processor, whether the candidate guide targets a common isoform and keeping the candidate guide for further selection if it meets a common isoform threshold;
determining, by the processor, whether the candidate guide has a precomputed prediction of editing outcomes and keeping the candidate guide for further selection if there is a precomputed prediction of editing outcomes;
determining, by the processor, whether the candidate guide maps to an expressed sequence and keeping the candidate guide for further selection if it maps to an expressed sequence;
determining, by the processor, a fraction of gene expression attributable to targeted transcripts and keeping the candidate guide for further selection if it meets a fraction of gene expression attributable to targeted transcripts threshold;
determining, by the processor, whether the candidate guide meets a common SNP overlap threshold and keeping the candidate guide for further selection if it meets an SNP overlap threshold;
determining, by the processor, which exon of the target gene is targeted and keeping the candidate guide for further selection if it meets a guide per exon threshold;
determining, by the processor, whether the candidate guide overlaps a selected guide and keeping the candidate guide for further selection if it meets a guide overlap threshold;
determining, by the processor, a predicted frameshift percentage for the candidate guide and keeping the candidate guide for further selection if it meets a predicted frameshift percentage threshold;
determining, by the processor, a GC content of the candidate guide and keeping the candidate guide for further selection if it meets a minimum GC content threshold;
determining, by the processor, the GC content of the candidate guide and keeping the candidate guide for further selection if it meets a maximum GC content threshold;
determining, by the processor, an off-target score for the candidate guide and keeping the candidate guide for further selection if it meets an off-target score threshold;
determining, by the processor, a position where the candidate guide targets a coding sequence and keeping the candidate guide for further selection if it meets a coding sequence position threshold; and
responsive to the candidate guide meeting the thresholds, selecting, by the processor, the candidate guide as a selected CRISPR guide.
